(19) United States
(12) Patent Application Publication
(10) Pub. No.: US 2010/0114063 A1
(43) Pub. Date: May 6, 2010

(54) CATHETER INJECTION MONITORING DEVICE

(75) Inventors: Daniel K. Recinella, Queensbury, NY (US); Leonard G.E. Schaefer, Queensbury, NY (US)

Correspondence Address: ANGIODYNAMICS, INC., 603 QUEENSBURY AVENUE, QUEENSBURY, NY, 12804, US

(73) Assignee: ANGIODYNAMICS, INC., Queensbury, NY (US)

(21) Appl. No.: 12/609,151

(22) Filed: Oct. 30, 2009

Related U.S. Application Data
(60) Provisional application No. 61/111,099, filed on Nov. 4, 2008.

(51) Int. Cl. A61M 25/00; G01M 19/00

(52) U.S. Cl. 604/508; 604/523; 73/865.8

(57) ABSTRACT

A catheter is provided that has at least one lumen which extends from a proximal end of the catheter to a distal end of the catheter. The catheter also has a catheter hub that has a body with a proximal end, a distal end, an exterior surface, and defining an interior cavity. The exterior surface of the body further defines at least one opening. At least one opening is defined therein the proximal end, and at least one opening is defined therein the distal end. The catheter also has a means for measuring and displaying the number of times that the catheter is injected with an infusate at a pre-determined pressure.

### CROSS-REFERENCE TO RELATED APPLICATIONS

This application claims priority to U.S. Patent Application Ser. No. 61/111,099, filed Nov. 4, 2008, which application is incorporated herein by reference in its entirety.

### FIELD OF THE INVENTION

The present invention relates to a medical device and method, and more particularly, a method for injecting fluid into a medical device and a blood vessel and a device for monitoring said injections.

### BACKGROUND OF THE INVENTION

Often PICCs (peripherally inserted central catheter (PICCs)) or central venous catheters (CVCs) are inserted into critically ill patients for the delivery of therapeutic solutions to assist in their treatment. PICCs typically provide short- or long-term peripheral access to the central venous system for intravenous therapy and blood sampling. A PICC is inserted into a peripheral vein, such as the cephalic vein, basilic vein, or brachial vein and then advanced through increasingly larger veins, toward the heart until the tip rests in the distal superior vena cava or cavo-atrial junction. In comparison, a central venous catheter (CVC) is placed into a large vein such as the internal jugular vein, the subclavian vein, or the femoral vein.

Typically, PICCs are made from a soft polymer material and are capable of withstanding the pressure from a slow infusion pump or gravity feed from a hanging bag. These pumps or bags generate low pressures and do not put excessive stress on the external walls of the catheter or, in the case of a multi-lumen catheter, on the septum between the lumens. Connecting these catheters to any type of power injector could cause the catheter to burst from the pressure of fluid delivery.

It is very common to have a need for obtaining images from patients that have various medical conditions (cancer, infections, etc.). The typical method for obtaining images, involves inserting a needle into a peripheral vein, as described above, and injecting dye. Placing a needle into a vein can be traumatic and painful for patients, as it puts the patient through another invasive medical procedure and also can destroy a vein that may be needed in the future for other medical interventions. It can also be time-consuming for medical personnel.

Another method for performing CT imaging with these patients is to perform a CT injection through an existing PICC or central line that is already in place inside of a blood vessel, avoiding the need for an additional access site. Catheters, such as PICCs, are unique because they may be used many times for high pressure CT injections. The ability to inject a dye through the catheter that is already in place saves time, money, and the patient from another medical procedure to place a catheter or needle only for dye injections. Contrast injections through central lines deliver contrast to the central circulation, thereby providing better mixing and better images with less total contrast delivered. Additionally, there are no shearing forces from the injection being applied to the walls of small veins. For these reasons it is desirable to use a central catheter vs. a peripheral needle for the patient's benefit.

By design, PICCs are made of materials that soften in the body. Materials that have a hardness greater than 95A durometer can cause phlebitis when in the vein for long periods of time. However, softer materials generally correspond with lower burst strengths and a propensity for showing material fatigue over time. Material fatigue occurs when a high stress is applied to the PICC wall and removed at varying intervals, which causes the walls of the PICC to be put into a cycle of tension and relaxation. This stress can be caused by a high pressure being applied to the catheter on a repeated basis, such as would be experienced with a CT injection. When the injection is complete, the stress on the wall dissipates.

If multiple injections are performed over time, the walls of the PICC tube experience a cycle of induced stress and relaxation. If the stress applied to the tube wall does not result in a stretching of the wall where plastic deformation occurs, the PICC catheter wall will return to its original dimensions when the stress is removed, but if the applied stress causes small, local plastic deformations, the catheter wall will not recover fully to its original dimensions. Cycles of induced stress and relaxation on a PICC occur when high pressure injections, like CT injections, are performed. The high pressure CT injections induce stress on the PICC, resulting in wall strain, or stretching, even at a very low level. As more stress is applied to and removed from the catheter, repeated injections over time will ultimately result in plastic deformation as the PICC catheter wall slowly becomes become thinner with each injection. This thin area is susceptible to bursting. The PICC will then eventually burst when under a high pressure injection.

Health care providers are familiar with the deleterious effects of multiple high pressure injections being performed repeatedly over the life of a PICC. PICCs that can sustain CT injections, at high flow rates and high pressures, have become popular in the market. Manufacturers may publish guidelines with their PICC products, stating the maximum number of injections their PICC can experience safely. However, there is concern that such PICCs may only be able to handle a finite amount of injections. There is currently no way of knowing how many injection cycles a PICC has experienced, or what limits the PICC can withstand. The average time a PICC is in a patient is 6 weeks, but can be as long as 52 weeks. Tracking the number of high pressure injections a PICC has experienced becomes an impossible task. If a PICC bursts during a high pressure injection, damage to the vessel wall may result. Being able to determine if a PICC is near its maximum number of injections would provide a level of safety for the patient and a comfort level for the physician. What is needed is a device that can be incorporated into a catheter, such as a PICC, to provide accurate readings of the number of times a catheter has been injected at a pre-determined maximum pressure to enable a user to determine the capability of the catheter to withstand additional injections at maximum pressures.

Without limiting the scope of the invention, a brief summary of some of the claimed embodiments of the invention is set forth below. Additional details of the summarized embodiments of the invention and/or additional embodiments of the invention may be found in the Detailed Description.

### BRIEF SUMMARY

A catheter having at least one lumen which extends from a proximal end of the catheter to a distal end of the catheter. The catheter also has a catheter hub that has a body with a proximal end, a distal end, an exterior surface, and defining an interior cavity. The exterior surface of the body further defines at least one opening. At least one opening is defined therein the proximal end, and at least one opening is defined therein the distal end. The catheter also has a means for measuring and displaying the number of times that the catheter is injected with an infusate at a pre-determined pressure. The means for measuring and displaying is positioned at least partially therein the interior cavity of the hub.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 illustrates a sectional view of the shaft of a vascular access catheter and catheter hub assembly.

FIG. 2 illustrates a cross-sectional view of the hub of FIG. 1 along line 2-2 of FIG. 1.

FIG. 3 illustrates a sectional view of an additional embodiment of the vascular access catheter shaft and catheter hub assembly.

FIG. 4 illustrates a cross-sectional view of the catheter hub assembly along line 4-4 of FIG. 3.

FIG. 5 illustrates a cross-sectional view of the catheter shaft along line 5-5 of FIG. 3.

FIG. 6 illustrates a sectional view of the catheter shaft with a wire and pressure transducer or sensor embedded within the catheter shaft wall.

FIG. 7 illustrates a sectional view of a monitoring gauge of the catheter hub assembly and one extension tube of the catheter.

FIG. 8 illustrates a sectional view of an alternative embodiment of the catheter hub assembly and one extension tube of the catheter.

FIG. 9 illustrates a sectional view of another alternative embodiment of the catheter hub assembly and one extension tube of the catheter.

FIG. 10 illustrates a sectional view of another alternative embodiment of the catheter hub assembly and one extension tube of the catheter.

FIG. 11 illustrates a sectional view of another alternative embodiment of the catheter hub assembly and one extension tube of the catheter.

FIG. 12 illustrates a sectional view of another alternative embodiment of the catheter hub assembly and one extension tube of the catheter.

FIG. 13 illustrates a sectional view of the monitoring gauge and an alternative embodiment of one extension tube of the catheter.

FIG. 14 is a sectional view of the monitoring gauge and an alternative embodiment of one extension tube of the catheter.

FIG. 15A is partial sectional view of another embodiment of the monitoring gauge and a partial cut-away view of the catheter hub.

FIG. 15B is a partial sectional view of the monitoring gauge after it has been inserted into the catheter hub.

FIG. 16A is another embodiment of the monitoring gauge and a partial cut-away view of the catheter hub.

FIG. 16B is another embodiment of the monitoring gauge after it has been inserted into the catheter hub.

FIG. 17 is a plan view of the proximal portion of the catheter with a monitoring gauge incorporated into a luer of the catheter.

FIG. 18 is a plan view of the proximal portion of the catheter with another embodiment of the monitoring gauge incorporated into a luer of the catheter.

FIG. 19A is a partial sectional view of the monitoring gauge and a partial cut-away view of the luer and one of the extension tubes.

FIG. 19B is a sectional view of the monitoring gauge and the catheter hub of FIG. 18.

FIG. 20 is a sectional view of the catheter and the catheter hub assembly incorporating an additional embodiment of a monitoring gauge.

FIG. 21 is an exploded view of another embodiment of the monitoring gauge of FIG. 20.

FIG. 22 illustrates a flowchart of a process of injecting a catheter with an infusate, using the monitoring gauge.

FIG. 23 illustrates a flowchart of a process of injecting a catheter with an infusate, using another embodiment of the monitoring gauge.

FIG. 24 illustrates a flowchart of a process of injecting a catheter with an infusate, using another embodiment of the monitoring gauge.

FIG. 25 illustrates a flowchart of a process of injecting a catheter with an infusate, using another embodiment of the monitoring gauge.

### DETAILED DESCRIPTION OF THE INVENTION

The following detailed description should be read with reference to the drawings, in which like elements in different drawings are identically numbered. The drawings, which are not necessarily to scale, depict selected preferred embodiments and are not intended to limit the scope of the invention. The detailed description illustrates by way of example, not by way of limitation, the principles of the invention.

The skilled artisan will readily appreciate that the devices and methods described herein are merely exemplary and that variations can be made without departing from the spirit and scope of the invention. It is also to be understood that the terminology used herein is for the purpose of describing particular embodiments only and is not intended to be limiting.

Ranges may be expressed herein as from “about” to one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. As used herein, the term “proximal” means closer to the operator while the term “distal” means further away from the operator than proximal.

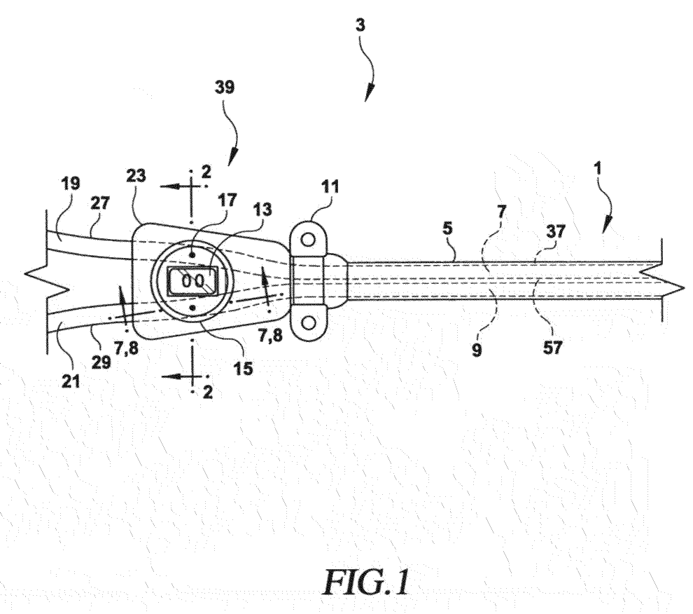

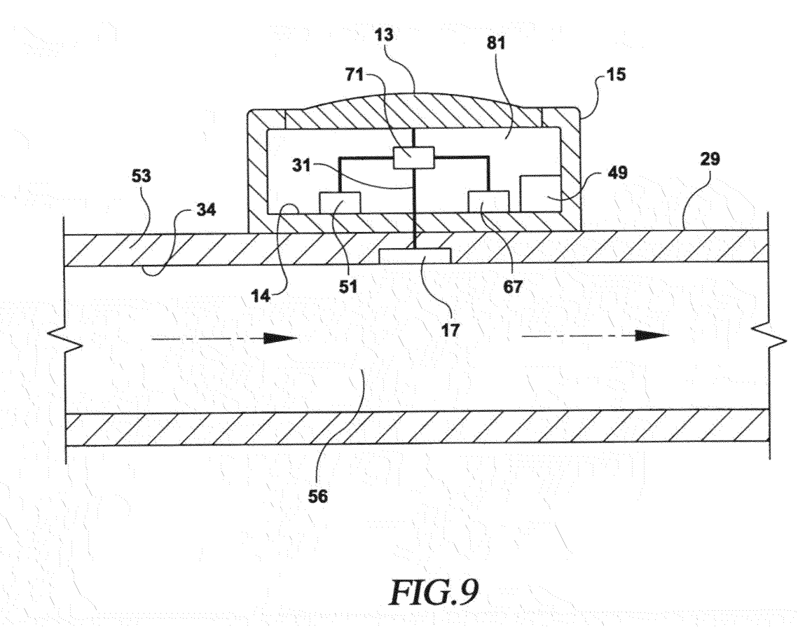

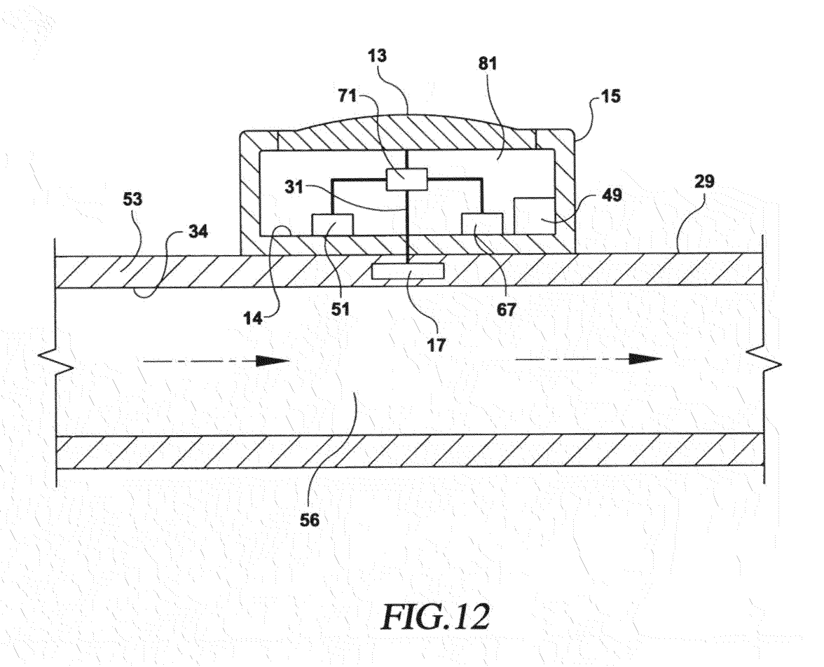

Referring now in detail to the drawings, in which like reference numerals indicate like parts or elements throughout the several views, in various embodiments, and referring to FIGS. 1-25, presented herein is an exemplary vascular access catheter, such as a peripherally inserted central catheter (PICC), having a catheter hub assembly, and a method of injecting a fluid, such as infusates or contrast agents, such as, for example and without limitation, drugs, such as urokinase or other anti-thrombotic agents, fluids, such as contrast media, under high pressure CT injection, into the vascular access catheter.

In FIG. 1, a proximal portion 3 of the catheter 1 having a catheter hub assembly 39 with a means for measuring and displaying the number of times that the catheter is injected with an infusate at a pre-determined pressure is illustrated. In one aspect, the means for measuring and displaying the number of times that the catheter is injected with an infusate at a pre-determined pressure can be a monitoring gauge 15. In one aspect, the monitoring gauge 15 can be defined therein at least a portion of a catheter hub 23. In one aspect, the hub 23 comprises a body having a proximal end, a distal end, an exterior surface, and the hub 23 also defines an interior cavity. In one aspect, the exterior surface of the hub body further defines at least one opening. In one aspect, the exterior surface of the hub body further defines a plurality of openings. At least one opening of the plurality of openings is defined therein the proximal end of the hub, and at least one opening of the plurality of openings is defined therein the distal end. In one aspect, the hub 23 surrounds the outer surface of the catheter shaft. In one aspect, the monitoring gauge 15 can be positioned at least partially therein the interior cavity of the hub 23. Although a dual lumen vascular access catheter is depicted, the vascular access catheter hub assembly 39 of the present invention can be used with any suitable single, dual, triple, or multiple lumen catheter. In one exemplary aspect, the monitoring gauge 15 can be used with a peripherally inserted central catheter (PICC). In one aspect, the monitoring gauge 15 can be used with any type of catheter, including, but not limited to, dialysis catheters. The monitoring gauge 15 is intended for use with single use, disposable catheters and for single patient use. The monitoring gauge 15 can comprise a housing having an interior cavity, a base and a wall extending therefrom the base and an outer surface that is configured for a leak-proof connection to an upper portion of the wall of the housing. In one aspect, a portion of a bottom surface of the monitoring gauge 15 and portions of the wall and base of the housing define the monitoring gauge 15. In one aspect, at least a portion of the outer surface of the monitoring gauge 15 is defined therein an opening of a plurality of openings of the body of the hub 23. In one aspect, the housing 15 preferably has a circular shape, as illustrated, although any suitable shape can be used.

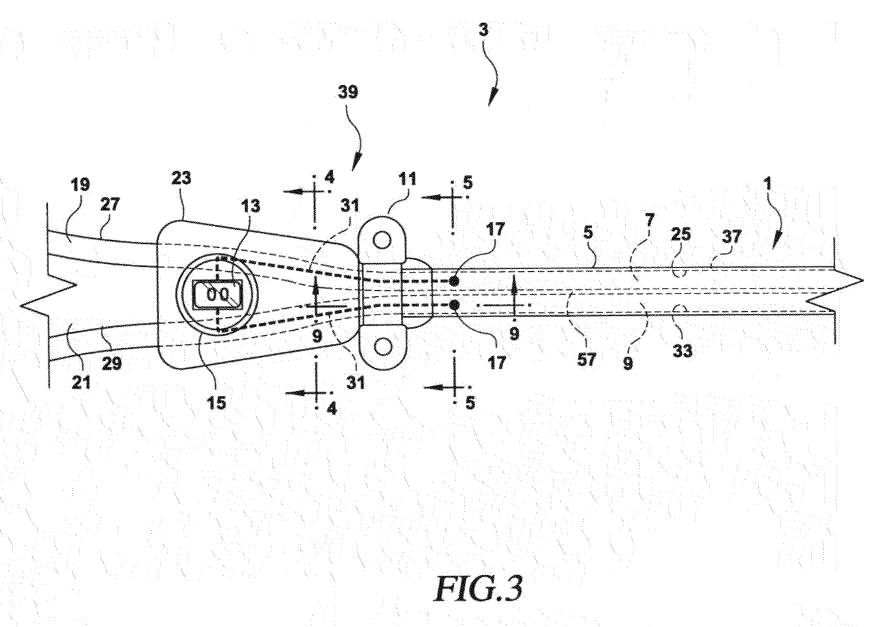

In one aspect, the monitoring gauge 15 can be used with a dual lumen catheter that has at least one extension tube having an outer wall, an inner wall, and at least one lumen. In one aspect, the catheter can comprise an extension tube 19 with outer wall 27 and an extension tube 21 with an outer wall 29, respectively. In one aspect, the catheter shaft comprises an outer surface and a plurality of lumens. The lumens extend from a proximal end of the catheter to a distal end of the catheter. In one aspect, at least a portion of the at least one lumen is fluidly joined within the hub 23 to at least a portion of at least one lumen of the catheter shaft. In one aspect, more particularly, the extension tubes 19, 21 are fluidly joined to the dual lumens 7, 9 of the catheter shaft 7 within the catheter hub 23 at a connection point (not shown). In one aspect, the monitoring gauge 15 can be in fluid communication with at least a portion of at least one extension tube lumen and at least one catheter lumen. In one aspect the monitoring gauge 15 and catheter disclosed herein can be configured to be used with at least one injection port or injection port for injections under high pressure, such as CT injections. In one aspect, the injection port is fluidly connected to at least one extension tube lumen. In one exemplary aspect, the at least one injection port can be designed for receiving fluid, such as contrast media. In another aspect, the at least one injection port can be designed for receiving saline and/or drugs. In one aspect, at least one supply port is designed for being connected to a power injector. The at least one injection port can be designed to be connected to at least one syringe. An injection port can be configured for connection to a high contrast CT injector. In one aspect, the catheter hub assembly 39 surrounds a catheter having a catheter shaft 1. The catheter shaft 1 has a catheter wall 37 with an outer surface 5 and an inner septum 57 that divides lumen 7 and lumen 9. In one aspect, as illustrated in FIGS. 3 and 5, lumen 7 has an inner wall 25, and lumen 9 has an inner wall 33. The catheter hub assembly 39 can also have suture wings 11. The monitoring gauge 15 has a compact design, such that it can fit snugly within at least a portion of the hub 23. In one aspect, the monitoring gauge 15 can have a diameter of up to approximately 0.5 inches and a height of up to approximately 0.25 inches, although other suitable dimensions can be used.

The monitoring gauge 15 is configured to be a means for measuring and displaying the number of times that the catheter can be injected with an infusate at a pre-determined pressure. In one aspect, the monitoring gauge 15 provides accurate, reliable, electronic readings of the number of times that a catheter has been injected with a fluid at a pre-determined pressure. In one exemplary aspect, the monitoring gauge 15 can be used with catheters that can withstand up to approximately 10 injections at maximum pressure of about 300 psi.

In one aspect, the monitoring gauge 15 comprises a pressure sensing means, such, as but not limited to, at least one pressure transducer or sensor 17 that can be used to sense the pressure in the lumens 55, 56 of the extension tube or the lumens 7, 9 of the catheter shaft 1. In one aspect, as illustrated, the pressure transducer 17 can be disposed therein a portion of the housing of the monitoring gauge 15 and can be adapted to produce electrical signals that are generated in response to fluid pressures within the lumens 55, 56, 7, 9. In one aspect, more than one pressure transducer 17 can be placed in communication with the lumens, as illustrated in FIGS. 1-5. In one aspect, at least one pressure transducer 17 can be placed in fluid communication with the lumens. In one aspect, two pressure transducers 17 can be used.

The monitoring gauge 15 can also comprise a display device, such as, but not limited to, an LCD (liquid crystal display) 13, as illustrated in FIGS. 1-5, for example. The LCD 13 can be an electo-optical amplitude modulator that is a thin, flat display device made up of any number of color or monochrome pixels arrayed in front of a light source or reflector that is an electroluminescent display. In one aspect, the display device can further comprise a visual indicator that is configured to provide data that represents the number of times the catheter has been injected at a pre-determined pressure. In one exemplary aspect, the visual indicator can be, but is not limited to, a digital numerical readout, at least one color, and a mechanical indicator. In one aspect, the LCD 13 is capable of providing a digital numerical readout of the number of times a catheter has been injected at a pre-determined pressure. In one exemplary aspect, the LCD 13 can provide numerical digital readouts from about 0 to about 99. In another embodiment, the LCD 13 can simply display a color, representing the number of injections done at a pre-determined maximum pressure. In one non-limiting example, when the catheter is injected at a pre-determined maximum pressure from about 0 to about 5 times, the LCD 13 will display a green light. In one exemplary aspect, when the number of injections is from about 5 to about 10, the LCD 13 will display a yellow light. When the number of injections is from about 10 to about 15, the LCD 13 will display a red light. In one alternative embodiment, the LCD 13 can be used display a color every time the catheter is injected at a pressure above a set amount, e.g. 100 psi, for example. In one aspect, the LCD 13 can be approximately 0.25 inches to approximately 0.375 inches in width.

The position of the monitoring gauge 15 within the at least a portion of the hub 23 of the catheter or the luer 43, as described below, is beneficial because it overcomes problems of sensors that have been used with catheters or other types of medical devices. Known sensors may be located at the tip of a catheter shaft or along the outside of the catheter shaft to sense the pressure in or around the catheter. These locations can be undesirable because they add bulk, can be more expensive, or can require additional equipment or components that must be connected to the pressure sensor, or they can be prone to breakdown or dislodgement. These sensors can also convey inaccurate data due to extraneous stresses or pressures such as catheter or patient movement, which can distort pressure readings. Other devices, such as pressure relief gauges, are known, which can encompass pressure relief balloons, valves, diaphragms, and other devices. However, these devices do not enable the user to gauge the number of times that a catheter has been injected at a certain pre-determined pressure. Furthermore, these devices can burst under high pressure, and are therefore subject to failure under high pressure injections, which can cause disruption to the catheter and to the patient. Such devices are also typically raised above the catheter and are not located within the catheter, which designs can be bulky and cumbersome. Such sensors also do not provide a means for determining the number of times a catheter has been injected under high pressures. In contrast, the present invention provides a built-in monitoring gauge 15 within the catheter hub assembly 39 that provides for a numerical indication of the number of times a catheter is injected with a fluid at a pre-determined pressure. Thus, the monitoring gauge 15 is beneficial because it allows hospitals to avoid purchasing separate components, such as pressure sensors and catheters, and it allows for safer procedures for the patient and the physician.

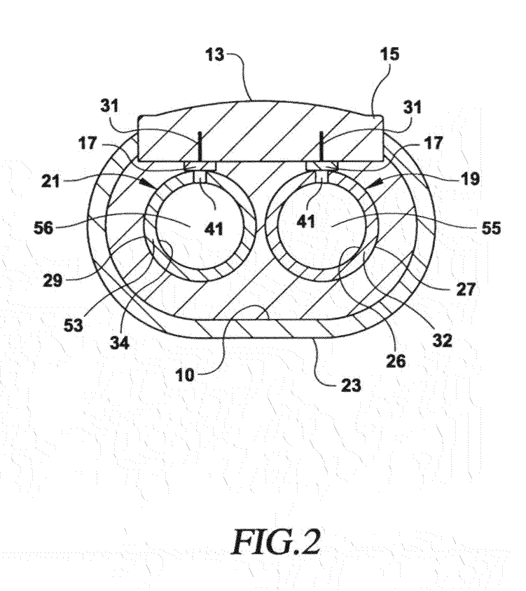

Referring to FIG. 2, a cross section of the catheter hub 23 is illustrated. The hub 23 comprises a body having a proximal end, a distal end, and an exterior surface. The hub comprises an interior cavity having a bottom surface 10. The exterior surface of the body further defines a plurality of openings. At least one opening of the plurality of openings is defined therein the proximal end, and at least one opening of the plurality of openings is defined therein the distal end of the hub 23. The monitoring gauge 15 is defined therein at least a portion of the catheter hub 23, and the LCD 13 is defined therein an exterior of the hub 23, such that it is visible to a practitioner. In one aspect, the LCD 13 can have a curved surface, as illustrated. In another aspect, the LCD 13 can have a flat surface, or any other suitable shaped surface. At least a portion of extension tubes 19 and 21, with outer walls 27 and 29, lumens 55, 56, and inner walls 26, 34, respectively, are positioned within the catheter hub 23. In one aspect, at least an outer surface of the pressure sensing means can be in fluid communication with at least one extension tube lumen via a channel 41. In one aspect, a channel 41 fluidly joins the extension tube lumens 55, 56 to at least a portion of an outer surface of the at least one pressure transducer or sensor 17. In one aspect, channel 41 can be an opening that is defined therein the entire thickness of the extension tube 19, 21 walls. The channel 41 extends from the inner walls 26, 34 of the extension tubes 19, 21 to the exterior surfaces 27, 29 of the extension tubes 19, 21. In one aspect, at least a portion of the outer surface of the pressure transducer 17 is secured to the outer surface 27, 29 of the extension tubes 19, 21. In one aspect, the monitoring gauge 15 can be positioned thereon at least a portion of the outer surface of at least one of the extension tubes 19 or 21 of the catheter. In one aspect, the monitoring gauge 15 can be positioned anywhere on the outer surface of the extension tubes 19, 21. In another aspect, at least a portion of the monitoring gauge can be positioned at least partially therein the interior cavity of the hub 23. In exemplary embodiment, at least a portion of the outer surface of the pressure transducer or sensor 17 can abut up against an outer bottom surface of the monitoring gauge 15.

In one aspect, at least one connecting wire 31 is positioned within at least a portion of the monitoring gauge 15 and connects the at least one pressure transducer or sensor 17 to the monitoring gauge 15 through an aperture (not shown) in a bottom surface of the monitoring gauge 15. When the monitoring gauge 15 is assembled, it provides a leak-free seal between the wiring 31 and the housing 15. In one aspect, the connecting wire 31 can be any suitable commercial wiring. In one aspect, the pressure transducer 17 can be in fluid communication with the extension tube lumens 55, 56 through channel 41. In one aspect, when fluid is injected into the lumens 55, 56, at least a portion of the outer surface of the at least one pressure transducer 17 comes in contact with the fluid, and the transducer 17 senses the fluid pressure. In one aspect, the pressure sensing means is capable of producing at least one electronic signal in response to fluid pressure inside the at least one extension tube lumen and the at least one catheter tube lumen. In one aspect, the transducer 17 can convert the fluid pressure into an electronic signal, which is transmitted to the LCD 13 via microprocessor 51 and a signal converter 71, described below, to generate a digital numerical readout of the increase in the number of fluid injections at a pre-determined maximum pressure.

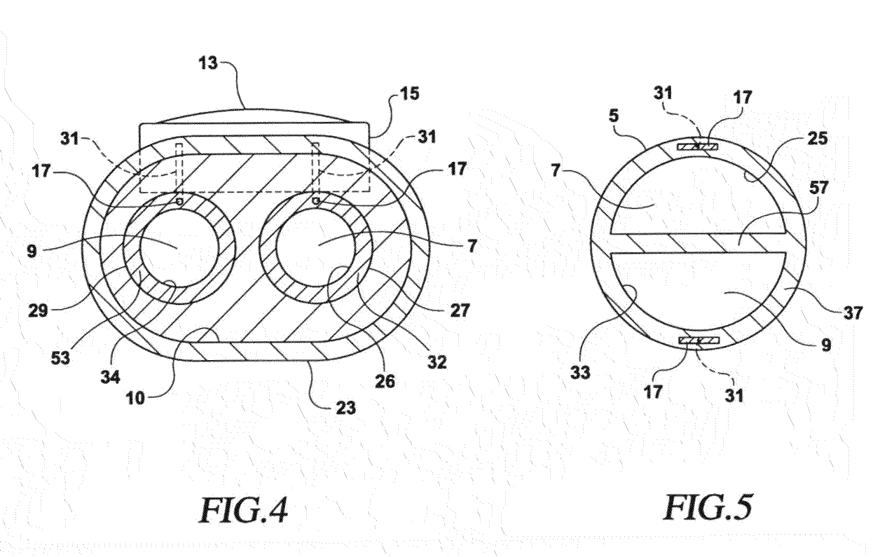

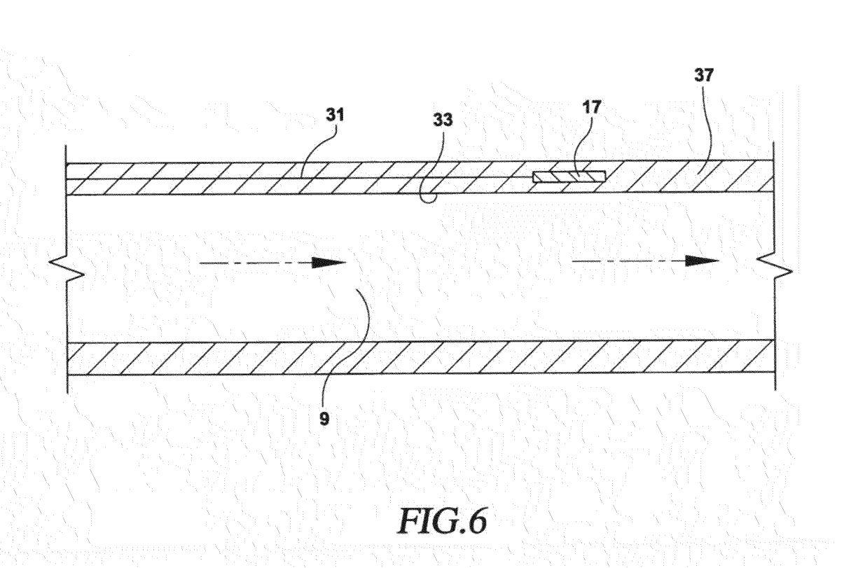

Another embodiment of the proximal end 3 of the catheter and the catheter hub assembly 39 is illustrated in FIGS. 3 through 6. In this aspect, the monitoring gauge 15 is defined therein a portion of the hub 23 and is configured for use with a dual lumen catheter, as described above. In this embodiment, connecting wires 31 can progress from the monitoring gauge 15 through at least one aperture in the bottom surface of the gauge 15 into at least one wall 53, 32 of the extension tubes 19, 21, and further into the wall 37 of the catheter shaft 1, such that they are embedded within the walls 53, 32 and 37, as illustrated in FIGS. 3 through 6. In the embodiment shown, at least one wire 31 can extend through each of the extension tube walls 53, 32 and the catheter lumen wall 37. In another aspect, more than two connecting wires 31 can be used. In one aspect, as illustrated in FIGS. 3 and 4, the wires 31 can extend through the catheter wall 37 and are electrically connected to the at least one pressure transducer or sensor 17. The transducers or sensors 17 are embedded within the catheter wall 37, at a position just distal of the catheter hub 23. In one aspect, the pressure transducers 17 can be positioned anywhere within the extension tube walls 53, 32 and the catheter wall 37. In one embodiment, the catheter shaft 1 can have markings (not shown) along the outside surface of the shaft 7 to indicate where the pressure transducer 17 is located within the catheter wall 37. In one exemplary aspect, the pressure sensing means can be positioned therein at least a portion of at least one extension tube and at least a portion of the catheter shaft such that the pressure sensing means does not occlude any portion of the extension tube lumens or the catheter lumens. More particularly, in one exemplary aspect, the at least one pressure transducer 17 can be embedded within at least a portion of the extension tube wall or the catheter wall 37 such that it does not occlude the extension tube lumens 55, 56 or the catheter lumens 7, 9, and it does not deter blood flow, as illustrated in FIG. 6. This configuration thereby decreases the chance of coagulation, thrombosis, fibrin buildup, infection, or other deleterious effects.

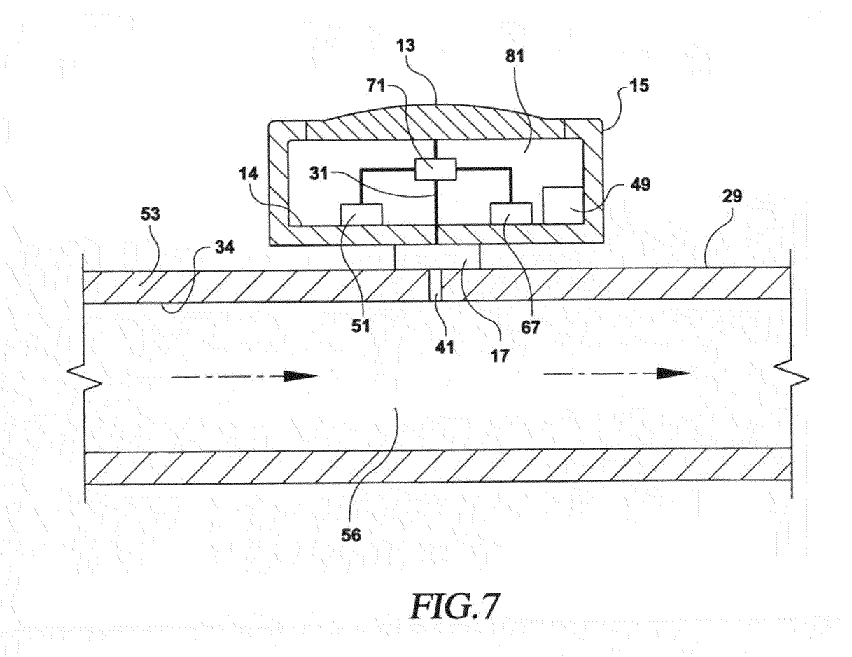

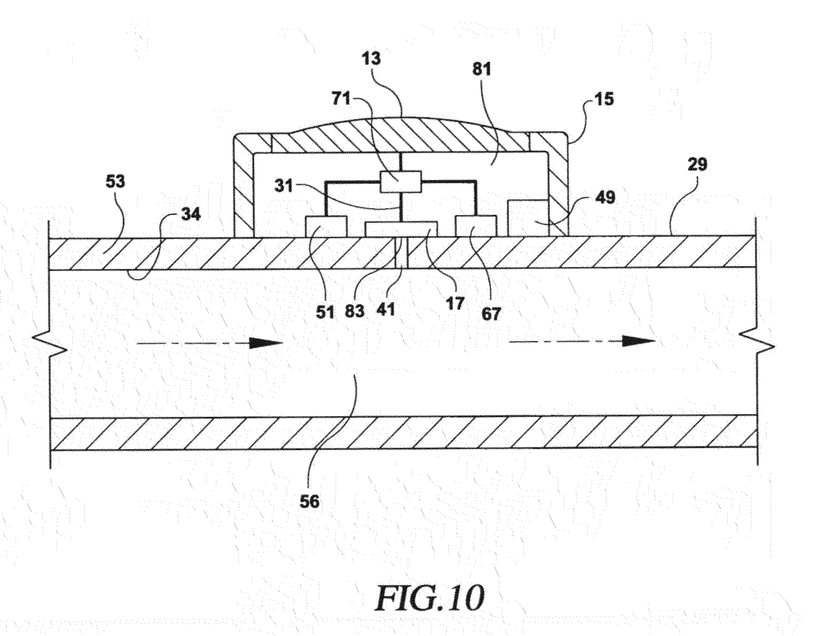

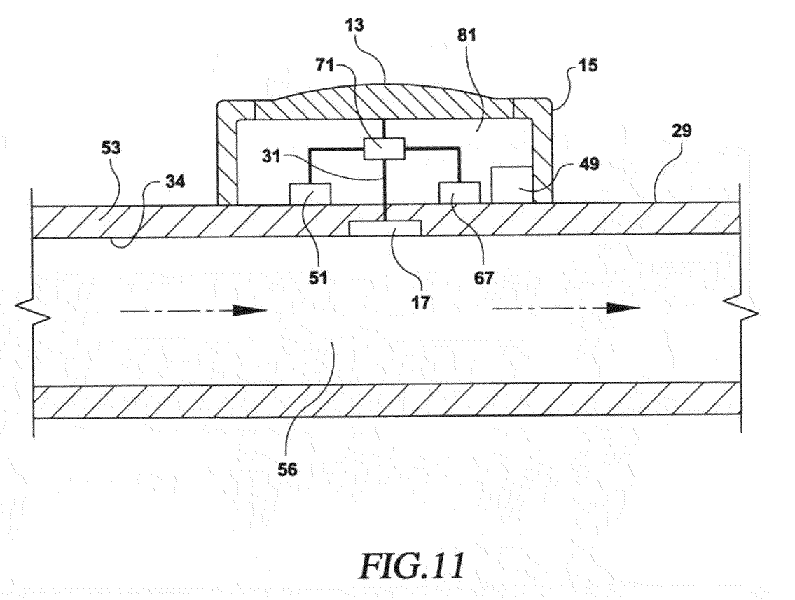

As illustrated in FIG. 7, a sectional view of one embodiment of the monitoring gauge 15 in relation to a portion of one of the extension tubes 21 is illustrated. In one aspect, the housing 15 can be a unitary piece having an inner wall 14 and an interior cavity 81. In one aspect, the housing 15 can be a unitary piece. In another aspect, the housing 15 can be composed of a top U-shaped portion and a bottom linear shaped portion or base that is joined together with the top portion to form one housing 15. In another aspect, the housing 15 can comprise just a top U-shaped portion, as illustrated in FIGS. 10 and 11. In another aspect, in FIGS. 10 and 11, the housing 15 can have no bottom wall portion, such that the electrical components of the housing 15 are in direct contact with the outer surface 29 of the extension tube 19. In one aspect, housing 15 is joined with LCD 13 to form a unitary piece. In one aspect the housing 15 can be comprised of, for example, stainless steel, lightweight titanium, polysulfone, polypropylene, polyethylene, polycarbonate, or any other suitable material that is biocompatible. Such materials are ideal to use for single-use applications in which a device is disposable.

In one aspect, the monitoring gauge 15 comprises an electronic assembly. The electronic assembly can comprise several electrical components, such as, but not limited to, a signal converter 71, microprocessor 51, pressure sensing means, such as, but not limited to, a transducer or sensor 17, battery 49, and optionally, an alarm mechanism 67, as illustrated in FIGS. 7-12. Any suitable commercially available electrical monitoring gauge 15 can be used. All or some of the electrical components of the assembly can be defined therein at least a portion of the interior cavity 81 of the hub 23. In one exemplary aspect, at least a portion of the electronic assembly can be disposed therein at least a portion of the housing such that the components are hermetically sealed in the housing 15, as illustrated in FIGS. 7, 9, 11, and 12. In one aspect, some, or all of the components of the electronic assembly can be secured to the inner surface 14 of the housing, as illustrated in various configurations in FIGS. 7-11. The monitoring gauge 15 is designed to be leak-proof such that the pressure transducer or sensor 17 is in communication with the fluid flowing through the lumen, but the fluid does not leak into the monitoring gauge 15.

The electrical assembly of the pressure monitoring gauge 15 has an electrical circuit for selectively outputting signals from the pressure transducer 17. In one aspect, the at least one pressure transducer 17 is connected via wires 31 to signal converter 71. In one aspect, signal converter 71 can also be connected to microprocessor 51 and alarm mechanism 67. One of ordinary skill in the art will recognize that the electrical components and configurations described herein are exemplary, and other electrical components and configurations can be used. In one aspect, the pressure transducer or sensor 17 is electrically or mechanically connected with LCD 13 through the wires 31 and the signal converter 71. In one aspect, at least a portion of the outer surface of the pressure transducer 17 can be exposed to the extension tube lumens 55, 56 or catheter lumens 7, 9. In one aspect, the at least one pressure transducer 17 can be capable of measuring pressure sensed within the lumens 55, 56 and 7, 9. In one aspect, the transducer 17 senses the internal fluid pressure of the lumens 55, 56 or 7, 9. The pressure transducer 17 communicates a signal which varies as a function of the fluid pressure within the lumens to a microprocessor 51, which interprets and sends the sensed pressure signal to a display means LCD 13 located on the pressure monitoring gauge 15 via a signal converter 51. In one aspect, the signal converter 51 contains electronic circuitry that converts an electrical signal from the transducer or sensor 17 to a digital signal form, and the LCD 13 displays this number on the LCD 13. In one aspect, after each injection into the catheter at a pre-determined pressure, the microprocessor 51 processes an increase in count, such that the LCD 13 displays a number “1” and so on.

In one embodiment, as illustrated in FIGS. 7, 9, 11, and 12, the transducer 17 can be positioned outside of the housing 15. In one aspect, as illustrated in FIGS. 2 and 7, an outer surface of at least a portion of the transducer 17 is positioned such that it is simultaneously in contact with the outer surface 29 at least one of the extension tubes 19, 21 and an outer surface of the monitoring gauge 15. In one aspect, the outer surface of the housing 15 is secured to at least a portion of the outer surface 29 of the extension tube. The at least one transducer or sensor 17 can be attached to the outer surface 29 of the extension tubes 19, 21 using any suitable attachment means. In one exemplary aspect, the at least one pressure transducer 17 can be mounted on a ceramic plate (not shown) that can be positioned such that it contacts at least a portion of the inner surface 14 of the monitoring gauge 15.

In another aspect, as illustrated in FIGS. 6, 9 and 11, the pressure sensing means 17 can be embedded within the extension tube walls 53, 32, such that at least a portion of an outer surface of the transducer 17 is flush with the inner surface 34, 26 of extension tube lumens 55, 56. Although the pressure sensing means is illustrated as being flush with the inner wall of the extension tube lumen, in one aspect, at least a portion of an outer surface of the pressure sensing means could be embedded within a catheter wall such that an outer surface of the pressure sensing means is flush with an inner wall of the catheter lumen. In one aspect, an outer surface of the transducer 17 can be fluidly joined to the lumen 56 of the extension tube 56 via channel 41, as illustrated in FIGS. 7, 8, and 10. In this aspect, at least a portion of the outer surface of the transducer 17 can be in fluid communication with the channel 41 through an opening 83 in the inner surface 14 of the housing 15, as illustrated in FIGS. 7, 8, and 10. In one aspect, a thin layer film can be positioned between the outer surface of the transducer 17 and the lumens 55, 56, 7, 9. This thin layer can have direct or indirect contact with the flow lumens 55, 56, 7, 9.

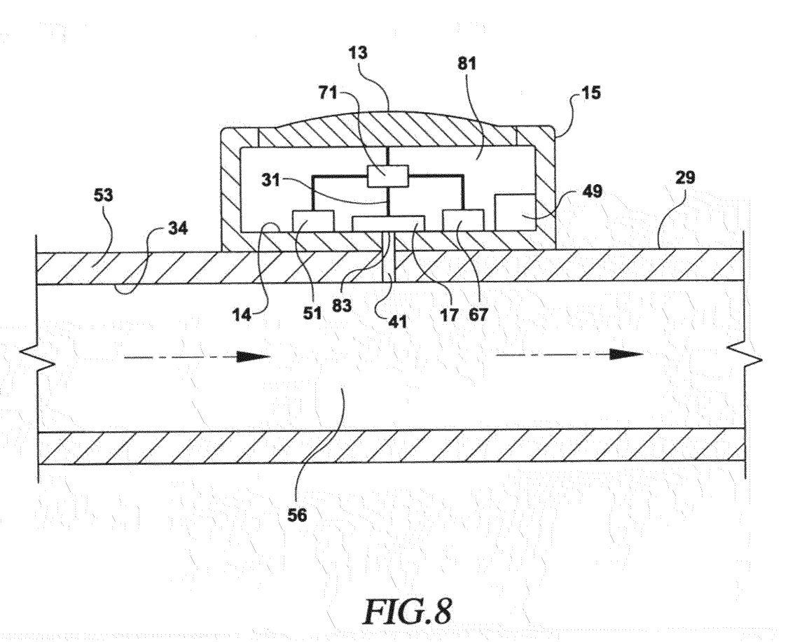

In another configuration, as illustrated in FIGS. 8 and 10, the transducer 17 can be defined therein at least a portion of the housing cavity 81. As illustrated in FIGS. 8 through 12, components 51, 67, and 49 can also be positioned within the housing 15, such that at least a portion of the outer surface of several components are in contact with the inner surface 14 of the housing 15. Although extension tube 19 is illustrated herein, the at least one pressure transducer 17 can be used with one or both of extension tubes 19, 21. In another aspect, as illustrated in FIGS. 8 and 10, the entire pressure transducer 17 can be defined therein at least a portion of the housing 15, such that at least a portion of the outer surface of the pressure transducer 17 can be in contact with the inner surface 14 of the housing 15. In one aspect, pressure transducer 17 can be secured to the bottom wall 14 of the housing 15. In another aspect, as illustrated in FIGS. 6, 9, 11, and 12, the pressure transducer 17 can be embedded within the catheter wall 37, such that the transducer is in indirect communication with the lumens 7, 9, 55, 56.

In one aspect, the pressure transducer or sensor 17 can be selected from any commercially available pressure sensing device, such as those sold by Lucas Novasensor (now GE Novasensor) or IC Sensors. Any suitable pressure transducer can be used. In one aspect, the pressure transducer 17 can be a piezoresistive pressure transducer or a silicon pressure transducer, or any of the like. In one aspect, the pressure transducer or sensor 17 can be coated with an anti-thrombogenic or anti-coagulating substance, such as heparin, or the like. In one-aspect, the pressure transducer 17 can pre-calibrated, which saves the practitioner time, thereby shortening procedure time.

In one aspect, the pressure transducer 17 can have a diaphragm (not illustrated), which responds to changes in fluid pressure and activates a mechanical pressure signal translation mechanism. The diaphragm can be disposed against a catheter wall so that as fluid flows through the catheter, the diaphragm senses fluid pressure changes without contact between the diaphragm and the fluid. When pressure acts on the diaphragm of the pressure transducer 17, it can cause a deflection of a piezoresistive element of the transducer 17, which can produce a signal which can be detected. The transducer or sensor 17 can produce an electronic signal that has a voltage level that is representative of the pressure inside the lumens 7, 9, 55, 56. A pressure sensitive element can be mounted on the diaphragm that provides output that is indicative of the pressure. In one aspect, the pressure transducer or sensor 17 can comprise a semiconductor integrated circuit chip that includes electronic components that form the transducer, which transducer includes a diaphragm. In one aspect, the diaphragm can be in fluid communication with the internal fluid pressure of the catheter lumen. More particularly, the diaphragm can be in fluid communication with the at least one catheter lumen and the at least one extension tube lumen, and the diaphragm can be selectively biasable in response to the fluid pressure that is generated within the at least one catheter lumen and the at least one extension lumen.

Optionally, alarm 67 can be defined therein a portion of the monitoring gauge housing 15. In one aspect, the monitoring gauge 15 can be pre-programmed with a pre-determined number of uses at a pre-determined maximum pressure, such that once the maximum number of injections is reached or exceeded, an alarm can sound, or a red or yellow light can appear. As the number of injections increases up to a pre-determined pressure, the alarm can go off, warning the practitioner that the catheter has reached its maximum number of injections at a pre-determined maximum pressure and that the catheter can no longer be used for high pressure injections, or the catheter needs to be replaced. In one aspect, the alarm 67 can be visual, auditory, tactile, color, or any other type of alarm, or a combination of several different alarms.

In one aspect, as illustrated in FIGS. 7 through 12, a small battery 49, such as a lithium battery, can be defined therein at least a portion of the housing 15. In one aspect the battery 49 can be used for providing power to the monitoring gauge 15. In one aspect the battery 49 can be a nanotechnology battery or any other suitable type of commercial battery, as known in the art. In one aspect the battery 49 can be capable of withstanding high pressures and can be capable of lasting up to approximately two years. The battery allows the monitoring gauge 15 to remain electrically active during the life of the battery.

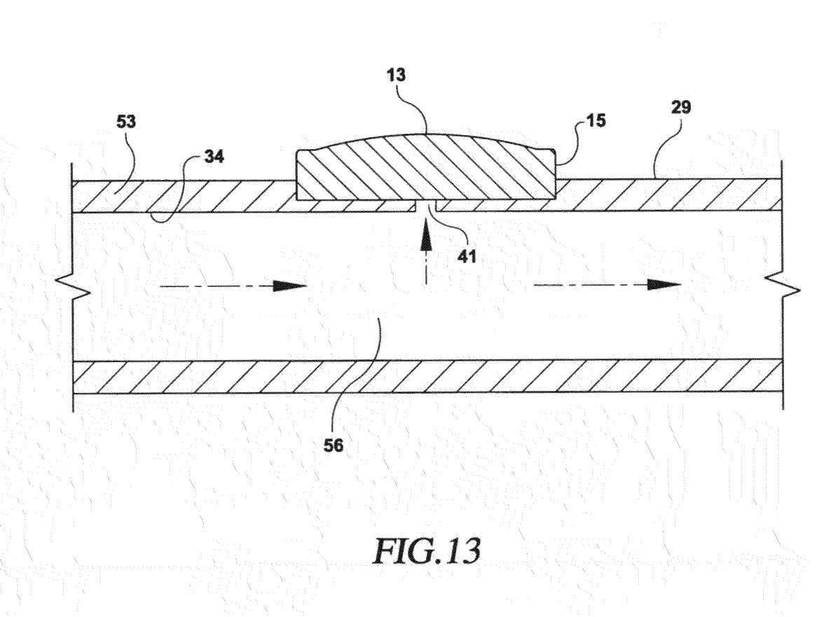

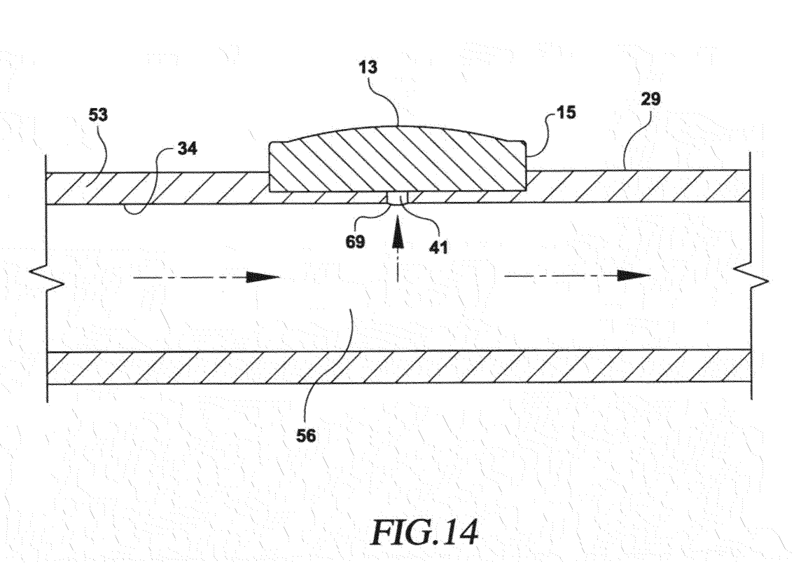

As illustrated in FIGS. 13 and 14, the channel 41 can be embedded within the wall of the extension tube 19 or 21. In another aspect, as illustrated in FIG. 13, a thin film or diaphragm 69 can be placed at the surface of the channel 41 between the lumen 56 of the catheter and the channel 41. The diaphragm 69 can be configured to flex or bend when it is in contact with the fluid that runs through the catheter lumen, as the pressure in the catheter lumen changes. The diaphragm 69 can have a membrane that is mechanically or electrically coupled to the pressure transducer 17. In one aspect, the diaphragm 69 can be biased outward toward the fluid, or the diaphragm can be planar and flat. In one aspect, the diaphragm 69 can be comprised of titanium. In another aspect, the diaphragm 69 can be comprised of a thin membrane, or the like. In one aspect, the pressure transducer or sensor 17 can have a membrane that is deformable upon contact with the fluid in the catheter lumen. The membrane can be composed of a flexible material such as, but not limited to, a rubber or polyurethane material. In one aspect, the membrane can be biased outwardly such that it has contact with a fluid in the catheter lumen. Upon contact with the fluid, the membrane can become inwardly biased in response to an increase in pressure.

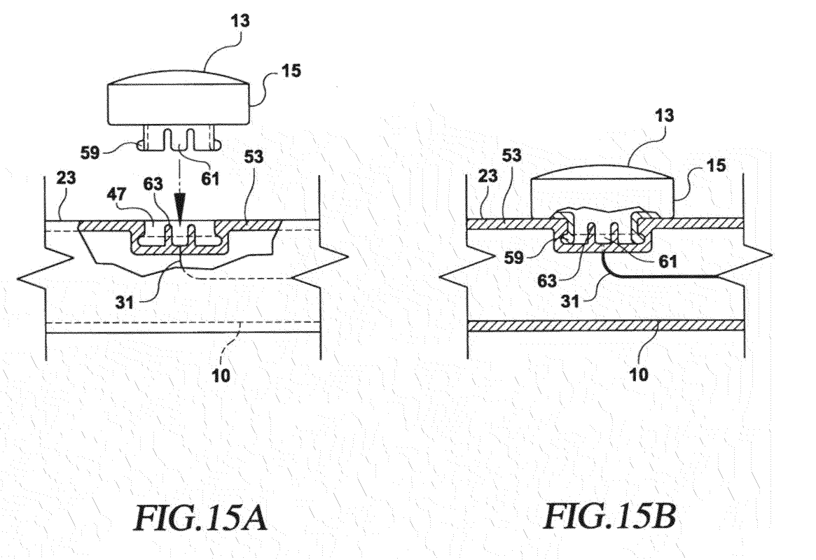

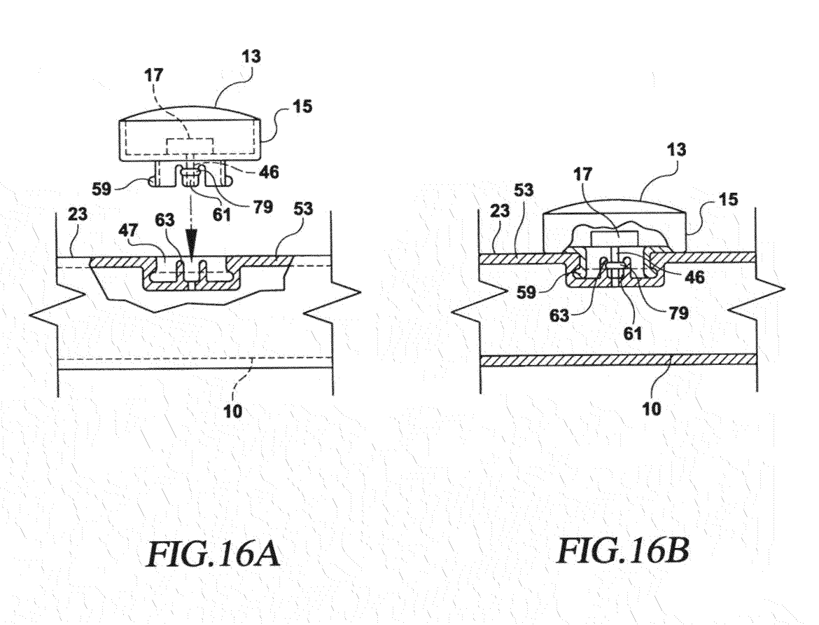

In one aspect, the monitoring gauge 15 is designed to be stationary. The monitoring gauge 15 is beneficial because it allows for inexpensive mass production and automation, and it can be pre-assembled with the catheter hub 23, as illustrated in FIGS. 1-8. Alternatively, the monitoring gauge 15 can be snap-fit into the hub 23, as illustrated in FIGS. 15A through 16B and 19A and 19B, such that it forms an interference fit or a press fit with the catheter hub 23. As illustrated in FIGS. 15A and 15B, the monitoring gauge 15 with LCD 13 can be inserted into the pocket 47 or removed using a snap-fit mechanism. In this aspect, the monitoring gauge 15 can comprise at least one extension tab 59. More particularly, the at least one extension tab 59 can be defined therein and extend from a bottom surface of the monitoring gauge 15, such that the at least one extension tab 59 is configured to fit into a pocket 47 that can be defined therein the hub 23 in a snap-fit mechanism. In one aspect, the tabs 59 can be used to fit into the pressure sensor pocket 47 in a locking fashion. In one aspect, the pressure sensor pocket 47 in the hub 23 can be an orifice that is configured for placement of the monitoring gauge 15 in the hub 23. In one aspect, the catheter can be manufactured with a pre-existing pocket 47. In one aspect, a male connector 61 can be positioned between the snap retainers 59, to allow the monitoring gauge 15 to be matingly joined with the female connector 63 in the pressure sensor pocket 47. Wiring 31 can be pre-positioned within the hub 23, such that when the monitoring gauge 15 is inserted into the pressure sensor pocket 47, the monitoring gauge 15 is electrically connected to the wiring 31. In another aspect, as illustrated in FIGS. 16A and 16B, the monitoring gauge 15 can be placed in the catheter hub 23. In one aspect, at least a portion of the monitoring gauge 15 can be placed within at least a portion of the interior cavity of the hub 23. In this aspect, the monitoring gauge 15 can have a sealing means which can be configured to be used to seal the monitoring gauge 15 into the pocket 47. In one aspect, the sealing means can be a mechanical seal. In another aspect, the seal can be an adhesive. In one aspect, the sealing means can comprise an O-ring 79. In one aspect, the O-ring can be circumferentially positioned around the male connector piece 61. The O-ring 79 can provide an interference fit between the male piece 61 and the female piece 63.

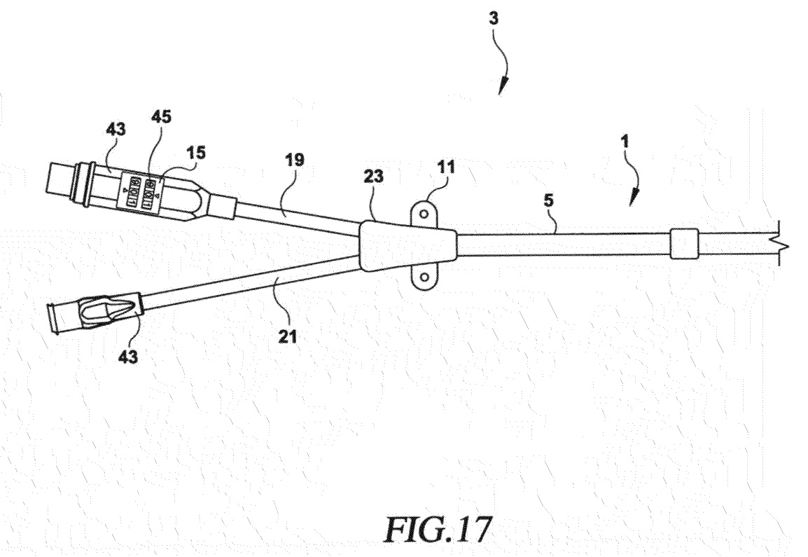

In one exemplary aspect, as illustrated in FIG. 17, monitoring gauge 15 can be defined therein at least a portion of the luer 43 and/or the extension tubes 19, 21. In one aspect, as illustrated in FIG. 17, the manual monitoring gauge 15 can have a usage indicator or counter 45 that is disposed therein at least a portion of the monitoring gauge 15, such that the indicator 45 is visible to a practitioner and is capable of providing a visual indication of the number of times that the catheter has been injected at a pre-determined maximum pressure. In one aspect, the luer 43 can have at least one usage indicator 45. In another aspect, the luer 43 can have two usage indicators 45. The at least one mechanical usage indicator 45 can be capable of providing a numerical readout of from one to three digits. In one aspect, the monitoring gauge 15 can have a square shape, as illustrated in FIG. 17, or any other suitable shape and dimensions. This manual monitoring gauge 15 is designed to be manually updated by a practitioner after each injection at a pre-determined pressure.

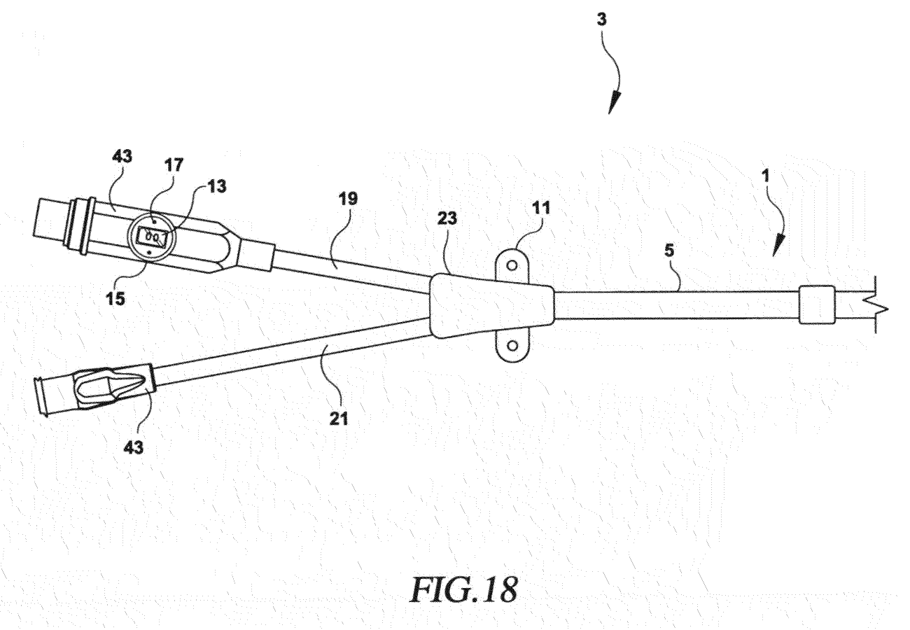

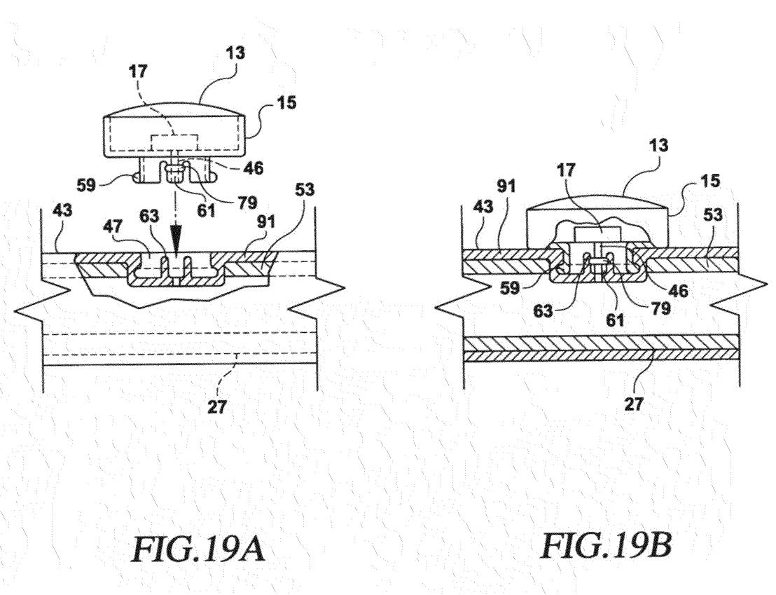

In another aspect, as illustrated in FIG. 18, at least a portion of the monitoring gauge 15, described above, can be positioned therein at least a portion of the luer 43. In one aspect, the monitoring gauge 15 can be pre-assembled with the catheter hub 23 such that it is positioned therein at least a portion of the catheter hub 23. In another aspect, as illustrated in FIGS. 19A and 19B, the monitoring gauge 15 can be snap-fit into an opening 47 in the wall of the luer 43 and extension tube 19, 21, as also described above in FIGS. 15A, B and 16A, B.

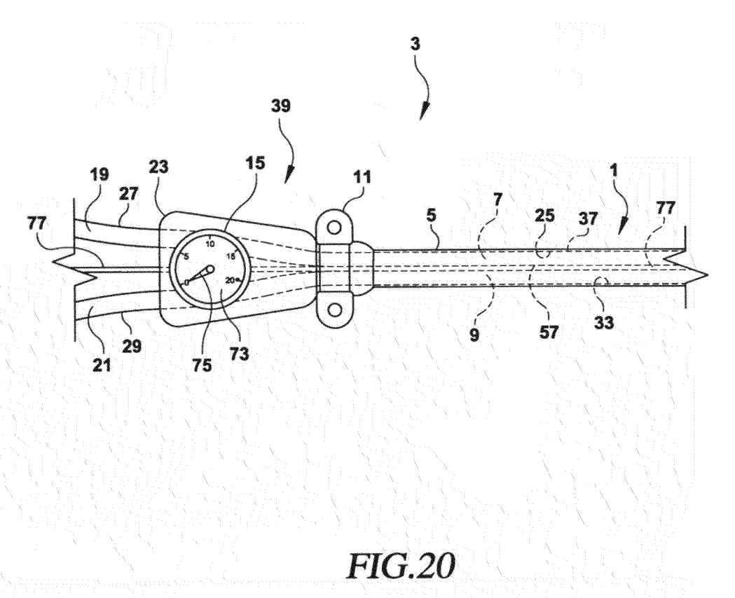

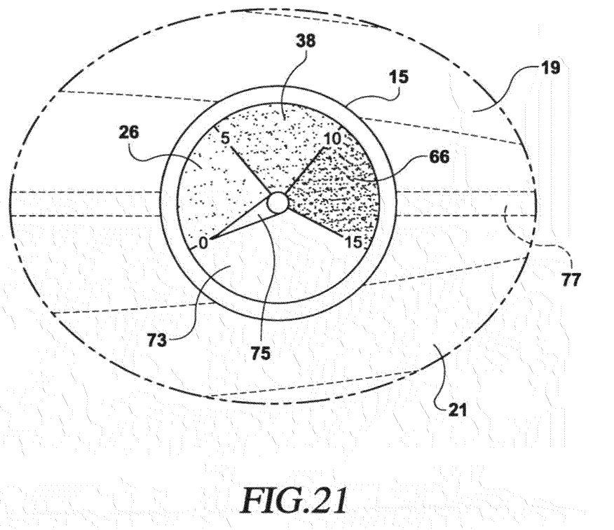

FIGS. 20 and 21 illustrate another embodiment of a monitoring gauge 15 for use with a catheter. In this aspect, the gauge is a mechanical or automated monitoring gauge 15 that can provide a numerical display in a dial format. In one aspect, the mechanical or automated monitoring gauge 15 has a mechanical usage indicator 75 that is disposed in at least a portion of the monitoring gauge 15 to provide a visual indication of the number of times that the catheter has been injected at a pre-determined maximum pressure, for example. The mechanical or automated monitoring gauge 15 produces a mechanical visual display, as illustrated in FIGS. 20 and 21, in which information is displayed and represented according to the physical position of at least one moving part, i.e., the indicator 75. In one aspect, the manual indicator 75 can be a freely-rotating pointer or dial that moves in response to each injection performed at a pre-determined pressure. In one aspect, the pointer 75 can move in a clockwise direction. In one aspect, the visual indicator 75 can rotate freely through a pre-determined angle that is determined by the fluid pressure magnitude. The pointer 75 can be rotated in varying increments, for example, 0 to 25, in relation to a display 73. In one aspect, the mechanical or automated monitoring gauge 15 can comprise a stepper motor that is electrically connected to the electronic circuitry within the monitoring gauge 15. The stepper motor can be incrementally adjusted to accurately display the number of amount of times that the catheter has been injected. In one aspect, a signal can be sent to the microprocessor 51 for another conversion into a digital signal by signal converter 71, which causes the stepper motor to move the manual indicator 75 on the gauge display 73. This enables the practitioner to view the number of times that the catheter has been injected at a pre-determined pressure. For example, after the catheter has been injected at a pre-determined pressure, the indicator 75 will increase from “0” to “1”, and so forth.

In another embodiment, the display 73 can have color-coded sections, such as illustrated in FIG. 21. In one exemplary aspect, from 0 to 5 injections, the visual indicator 45 can remain in the green portion 26 of the display 73, indicating that the number of injections at a pre-determined maximum pressure has not been reached. In another aspect, from 5 to 10 injections, the visual indicator 45 can be positioned in the yellow portion 38 of the display 73, which color functions as a warning that the maximum number of injections at a maximum pre-determined pressure is near. In another aspect, when the maximum number of injections has been reached, the visual indicator 75 can be positioned within the red region 66, which indicates to a practitioner that high pressure injections at a pre-determined pressure should be stopped. In one aspect, the monitoring gauge 15 can be pre-calibrated so that when a certain number of injections have been completed at a pre-determined maximum pressure, the visual indicator 75 will be positioned in the colored region 66, and/or the alarm 67 will sound.

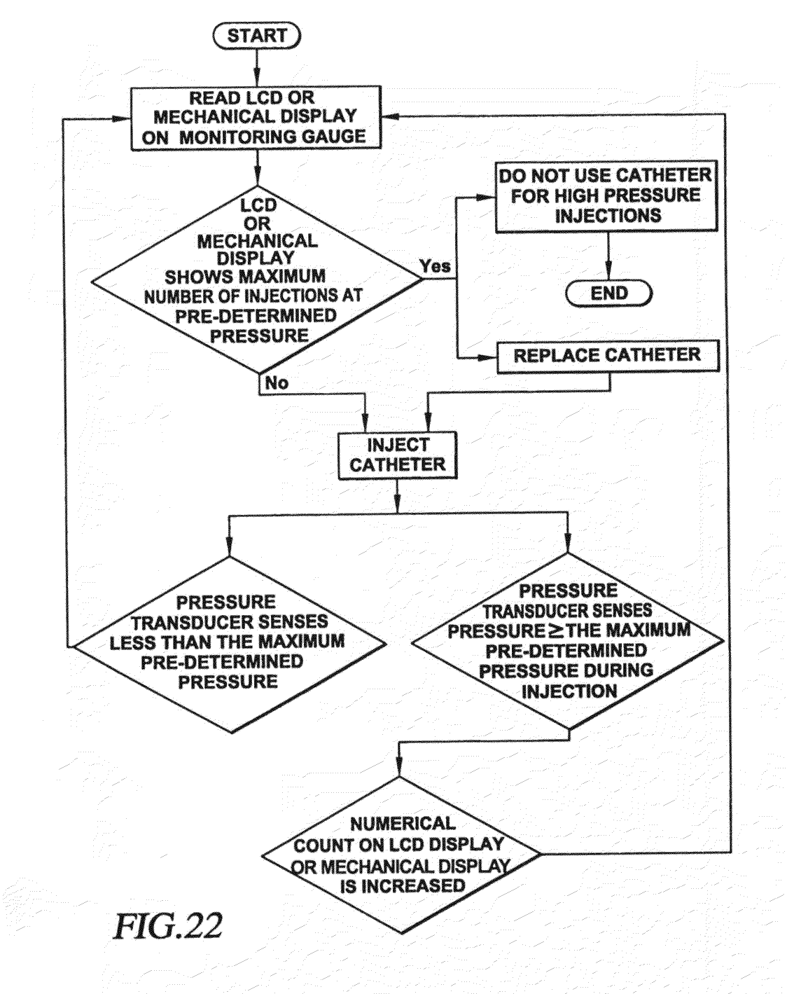

In one aspect, a method of injecting the catheter with an infusate, such as a contrast agent or other fluid, under high pressure, is illustrated. More particularly, the method involves injecting an infusate into a patient's body by providing a catheter having at least one display device as described herein and inserting at least a portion of the catheter into a patient's body. Then a practitioner begins by reading or visually perceiving information displayed in the display device, confirming the number of times that the catheter has been injected at a pre-determined pressure, injecting the infusate into the plurality of lumens, and selectively repeating the steps of visually perceiving, confirming, and injecting, until the catheter has been injected a pre-determined number of times at a pre-determined maximum pressure. In one exemplary aspect, as illustrated in FIG. 22, a practitioner reads or visually perceives the LCD 13 of the electrical monitoring gauge 15 or the display 73 of the mechanical monitoring gauge 15, illustrated in FIGS. 1-16B and 18-20. In one exemplary aspect, when an injection of a pre-set pressure minimum is experienced (e.g., 100 psi), the LCD 13 will display the number “1”, or the indicator 75 on the display 73 will move such that it points to the number “1”, as described above. After a subsequent infusate injection is completed, under high pressure, for example, the LCD 13 will display a “2” and then “3” and so on, or the indicator 75 will point to “2” or “3”. If the maximum number of injections has not been reached, then the practitioner can inject the catheter again under high pressure. After the catheter is injected, the pressure transducer 17 can sense less than the maximum pre-determined pressure, in which case, the practitioner will read the LCD 13 or display 73, and inject the catheter again. If the pressure transducer 17 senses a pressure that is equal to or greater than a maximum pre-determined pressure, the LCD 13 will increase by one, or the indicator 75 will move by one. This process can continue until the LCD 13 or display 73 shows that the maximum pre-determined number of injections at a maximum pre-determined pressure has been reached. In this instance, the practitioner will know that the catheter can no longer be used for high pressure injections, and the injections will end. In an alternative aspect, the catheter could be replaced, after which the catheter can be injected again with an infusate at a pre-determined high pressure. In one aspect, the maximum pressure threshold of the catheter can be pre-set to a predetermined level. For example, once the pressure reaches 100 psi, an alarm can go off, in conjunction with the numerical displays of the monitoring gauge 15.

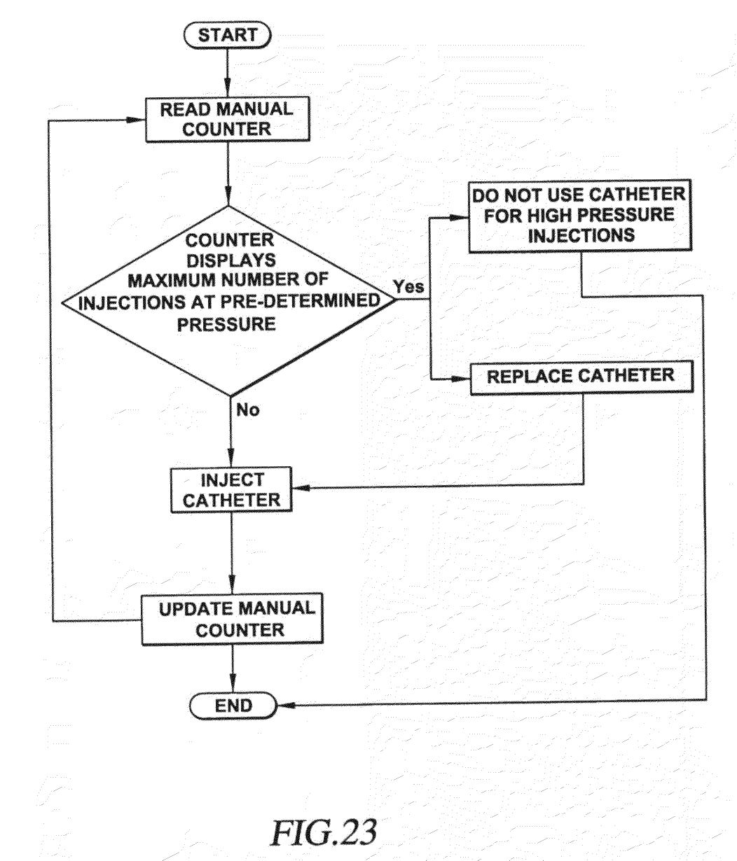

FIG. 23 illustrates a method of injecting the catheter with a fluid, such as, but not limited to, an infusate, such as a contrast agent or other fluid, under high pressure in conjunction with the manual monitoring gauge 15, described in FIG. 17. As illustrated in FIG. 23, a practitioner begins by reading the manual counter display 45 of the manual monitoring gauge 15. If the manual counter 45 does not display the maximum number of injections at a pre-determined pressure, then the practitioner can inject the catheter at a pre-determined pressure. In one exemplary aspect, when an injection of a pre-set pressure minimum is completed (e.g., 100 psi), the practitioner will manually move the counter display 45 upward by one numerical digit, such that it displays the number “1”, and so on. This process can continue until the manual counter display 45 displays the maximum number of injections at a maximum pre-determined pressure, which will indicate to the practitioner that the catheter can no longer be used for high pressure injections, at which point the injections will end. Alternatively, the catheter can need to be replaced, after which the catheter will be injected again under high pressure until the maximum number of injections at high pressure has been reached.

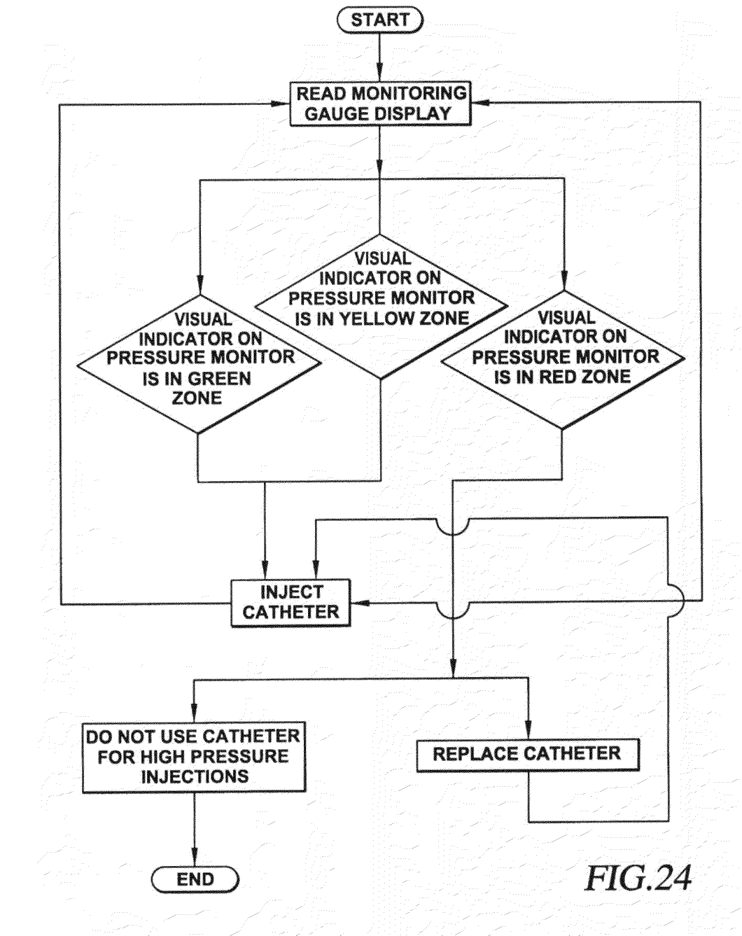

Another method of injecting the catheter with a fluid, such as, but not limited to, an infusate, such as a contrast agent or other fluid, under high pressure, is illustrated in FIG. 24, using the monitoring gauge 15, illustrated in FIG. 21. In this method, the practitioner begins by reading the monitoring gauge 15 display 73. If the maximum number of injections at a pre-determined maximum pressure has not been reached, the visual indicator 75 will remain in the green zone 26. The practitioner can then inject the catheter at a pre-determined maximum pressure, for instance, e.g., 100 psi. The practitioner will then read the monitoring gauge display 73 again and repeat this process until the visual indicator 75 moves to the yellow zone 38. The placement of the visual indicator 75 in the yellow zone is an indication to the practitioner that the maximum number of injections at a pre-determined maximum pressure is near. If the visual indicator 75 is positioned in the red zone 66, then the practitioner will know that high pressure injections into the catheter must be stopped, or the catheter needs to be replaced, after which injections can be continued until the maximum number of high pressure injections has been reached.

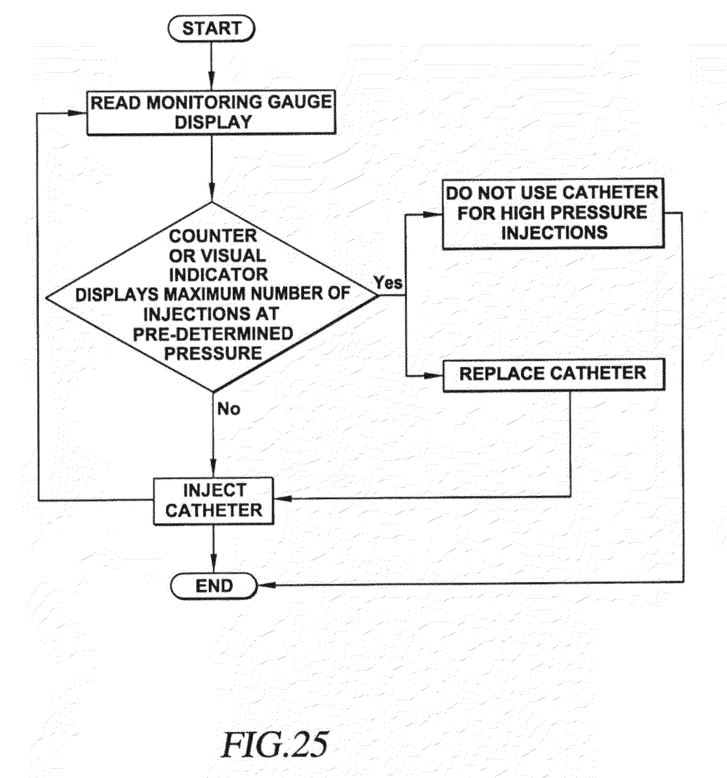

FIG. 25 illustrates a method of injecting the catheter with an infusate, such as a contrast agent or other fluid, under high pressure, using the mechanical monitoring gauge 15, as illustrated in FIG. 20. A practitioner begins by reading the display 73 of the mechanical monitoring gauge 15. If the maximum number of injections has not been reached, then the practitioner can inject the catheter under high pressure. In one exemplary aspect, after an injection of a pre-set pressure maximum is experienced (e.g., 100 psi), the visual indicator 75 will move such that it is pointing to the number “1”. The practitioner will then read the monitoring gauge display 73 and continue infusate injections at a pre-determined pressure until the maximum number of injections at a pre-determined high pressure has been reached, at which point the practitioner will stop using the catheter for high pressure injections or replace the catheter and continue to inject the catheter under high pressure.

One of ordinary skill in the art would understand that the method of injecting a catheter with an infusate can be done with any type of catheter. In one exemplary embodiment, the method could involve determining the number of injections into a catheter of a fluid at a pre-determined pressure using any of the following steps: providing a catheter, inserting a catheter into a patient, injecting a catheter with a fluid, and confirming the number of times that the catheter has been injected at a pre-determined pressure. The fluid could be an infusate, as described above. Additionally, in one aspect, this method of using any catheter in the method of determining the number of injections at a pre-determined pressure, could involve any of the steps described above.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. The words “including” and “having,” as used herein including the claims, shall have the same meaning as the word “comprising.” Those familiar with the art can recognize other equivalents to the specific embodiments described herein, which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the selected embodiments of the invention. Those skilled in the art can recognize other equivalents to the specific embodiments described herein which equivalents are intended to be encompassed by the claims attached hereto.

What is claimed is:

1. A catheter, comprising:
a catheter shaft having an outer surface and at least one lumen, wherein the at least one lumen extends from a proximal end of the catheter to a distal end of the catheter;
a catheter hub comprising a body having a proximal end, a distal end, an exterior surface, and defining an interior cavity, wherein the exterior surface of the body further defines at least one opening, wherein the at least one opening is defined therein the proximal end, and the at least one opening is defined therein the distal end, wherein the hub surrounds the outer surface of the catheter shaft; and
a means for measuring and displaying the number of times that the catheter is injected with an infusate at a pre-determined pressure.

2. The catheter of claim 1, wherein the catheter further comprises at least one extension tube having an outer wall and an inner wall and at least one lumen, wherein at least a portion of the at least one lumen is fluidly joined within the hub to at least a portion of at least one lumen of the catheter shaft.

3. The catheter of claim 1, wherein the means for measuring and displaying comprises a housing having an interior cavity, a base and a wall extending therefrom the base and an outer surface that is configured for a leak-proof connection to an upper portion of the wall of the housing.

4. The catheter of claim 3, wherein the means for measuring and displaying further comprises at least one extension tab and at least one sealing means, wherein the at least one extension tab is defined therein and extends from a bottom surface of the means for measuring and displaying, such that the at least one extension tab is configured to fit into a pocket defined therein the hub in a snap-fit locking fashion, and wherein the sealing means can be configured to seal the means for measuring and displaying into the pocket.

5. The catheter of claim 2, wherein the means for measuring and displaying is positioned thereon at least a portion of the outer surface of the at least one extension tube of the catheter and at least partially therein the interior cavity of the hub.

6. The catheter of claim 1, wherein the catheter further comprises a luer, and wherein the means for measuring and displaying is defined therein at least a portion of the luer.

7. The catheter of claim 2, wherein the means for measuring and displaying is in fluid communication with at least a portion of at least one catheter lumen and at least one extension tube lumen.

8. The catheter of claim 2, wherein the means for measuring and displaying is configured for use with at least one injection port, wherein the injection port is fluidly connected to at least one extension tube lumen, and wherein the injection port is capable of being used under high pressure.

9. The catheter of claim 7, wherein the means for measuring and displaying further comprises an electronic assembly, and wherein the electronic assembly comprises at least a signal converter, a microprocessor, a battery, and an alarm mechanism.

10. The catheter of claim 9, wherein at least a portion of the electronic assembly is disposed therein at least a portion of the interior cavity of the hub.

11. The catheter of claim 9, wherein the electronic assembly further comprises a pressure sensing means.

12. The catheter of claim 11, wherein at least a portion an outer surface of the pressure sensing means is in fluid communication with at least one extension tube lumen via a channel.

13. The catheter of claim 11, wherein the pressure sensing means is positioned therein at least a portion of at least one extension tube and at least a portion of the catheter shaft such that the pressure sensing means does not occlude any portion of the extension tube lumens or the catheter lumens.

14. The catheter of claim 13, wherein the pressure sensing means is embedded within at least a portion of at least one of the extension tube wall or the catheter wall such that at least a portion of an outer surface of the pressure sensing means is flush with the inner surface of at least one extension tube lumen.

15. The catheter of claim 11, wherein the pressure sensing means is capable of producing at least one electronic signal in response to fluid pressure inside the at least one extension tube lumen and the at least one catheter lumen.

16. The catheter of claim 15, wherein the pressure sensing means further comprises a diaphragm that is in fluid communication with the at least one catheter lumen and the at least one extension tube lumen, and wherein the diaphragm is configured to be selectively biasable in response to the fluid pressure.

17. The catheter of claim 1, wherein the means for measuring and displaying further comprises at least one display device.

18. The catheter of claim 17, wherein the at least one display device further comprises a visual indicator that is configured to provide data that represents the number of times the catheter has been injected at a pre-determined pressure.

19. The catheter of claim 18, wherein the visual indicator is selected from the group comprising a digital numerical readout, at least one color, and a mechanical indicator.

20. A method of injecting an infusate into a patient's body, wherein the method comprises:
providing a catheter comprising
at least one lumen, wherein the at least one lumen extends from a proximal end of the catheter to a distal end of the catheter;
a catheter hub comprising a body having a proximal end, a distal end, an exterior surface, and defining an interior cavity, wherein the exterior surface of the body further defines at least one opening, wherein at least one opening is defined therein the proximal end, and at least one opening is defined therein the distal end; and
a means for measuring and displaying the number of times that the catheter is injected with an infusate at a pre-determined pressure, wherein the means for measuring and displaying further comprises at least one display device;
inserting at least a portion of the catheter into the patient's body;
visually perceiving information displayed in the at least one display device;
confirming the number of times that the catheter has been injected at a pre-determined pressure; and
injecting the infusate into the at least one lumen.

21. The method of claim 20, wherein the method further comprises selectively repeating the steps of visually perceiving, confirming, and injecting until the catheter has been injected a pre-determined number of times at a pre-determined pressure.

22. The method of claim 20, wherein the step of injecting an infusate into the catheter further comprises injecting the infusate under high pressure.

23. The method of claim 20, wherein the method further comprises replacing the catheter with a new catheter.

24. A method of determining the number of injections into a catheter of a fluid at a pre-determined pressure, wherein the method comprises:
injecting a catheter with a fluid;
confirming the number of times that the catheter has been injected at a pre-determined pressure.
